(19) United States
(12) Patent Application Publication
(10) Pub. No.: US 2006/0147501 A1
(43) Pub. Date: Jul. 6, 2006

(54) Collagen compositions and biomaterials

(76) Inventors: Patrick J. Hillas, San Francisco, CA (US); James W. Polarek, Sausalito, CA (US); Chunlin Yang, Bellemead, NJ (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(21) Appl. No.: 10/546,489

(22) PCT Filed: Feb. 27, 2004
(86) PCT No.: PCT/US04/05966

§ 371 Date: Aug. 22, 2005

Related U.S. Application Data
(60) Provisional application No. 60/450,989, filed on Feb. 28, 2003.
(60) Provisional application No. 60/510,619, filed on Oct. 10, 2003.

(51) Int. Cl. A61K 9/70; A61K 38/17

(52) U.S. Cl. 424/443; 530/356

(57) ABSTRACT

The invention relates to biomaterials and, in particular, biomaterials containing collagen.

This application claims the benefit of U.S. Provisional Application Ser. No. 60/450,989, filed on 28 Feb. 2003, and U.S. Provisional Application Ser. No. 60/510,619, filed on 10 Oct. 2003, each of which is incorporated by reference herein in its entirety.

Portions of this work were supported by RO1 grant AR45879 from the National Institutes of Health.

### FIELD OF THE INVENTION

The present invention relates to collagen compositions and biomaterials and to uses of collagen compositions and biomaterials in various biomedical applications.

### BACKGROUND OF THE INVENTION

Biomaterials, used in various medical applications as or in medical devices, contact the living cells, tissues, or organs, or fluids of a patient as part of their use and performance. Biomaterials can include metals and alloys, glasses and ceramics, natural or synthetic polymers, biomimetics, composites, and/or naturally derived or engineered materials. Biomaterials have been central to recent advances in the areas of tissue engineering, drug delivery, and implantable devices.

Collagen, a matrix protein, has been used widely in the biomaterials area, appearing in or as various matrices, membranes, sponges, scaffolds, stents, and other devices, implanted or applied. Collagen's structural and functional properties are uniquely suited to these diverse applications. For example, collagen is useful in tissue engineering procedures in which an implanted device serves to guide proper tissue regeneration, providing structural support and a suitable surface for cell and tissue growth/regrowth. Collagen's absorbable properties minimize the likelihood of infections and other downstream adverse immunological reactions associated with the implanted material. Collagen is hemostatic, making it suitable for use in medical sponges, bandages, dressings, sutures, etc. Collagen facilitates wound healing, tissue regeneration, etc., by providing sites for cell attachment and migration. Collagen's three-dimensional structure permits effective drug and nutrient exchange with the surrounding environment and prevents build-up of waste products, etc., enabling its use in various drug delivery devices and systems, facilitating cell/tissue growth/regrowth in engineering applications, etc.

Therefore, there is a need in the art for biomaterials comprising collagen and capable of offering improved performance in the wide range of applications in which biomaterials are used. The present invention meets this need by providing biomaterials containing collagen and having specifically defined structural and functional features, e.g., surface area, tensile strength, denaturation temperature, cell density, collagenase resistance, etc

### SUMMARY OF THE INVENTION

The present invention relates to biomaterials and, in particular, to biomaterials containing collagens. In certain embodiments, the collagen is recombinant collagen, human collagen, or recombinant human collagen, respectively. In various embodiments, the collagen is selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII. In some embodiments, the collagen is collagen of one collage type free of any other collagen type; in other embodiments, the collagen is a specified or unspecified mixture of more than one collagen type. It is specifically contemplated that in some embodiments, the biomaterial is a biomaterial selected from the group consisting of sponges; matrices; membranes; sheets; implants; scaffolds; barriers; stents; grafts, e.g., a tissue graft; sealants, e.g., vascular sealants, tissue sealants, etc.; corneal shields; artificial tissues, e.g., artificial skin; hemostats; bandages; dressings, e.g., wound dressings; coatings, e.g., stent coatings, graft coatings, etc.; adhesives; sutures; and drug delivery devices. It is further contemplated that these biomaterials can be used in various applications and procedures, including, but not limited to, the following: tissue engineering, tissue augmentation, guided tissue regeneration; drug delivery; various surgical procedures including restorative, regenerative, and cosmetic procedures; vascular procedures; osteogenic and chondrogenic procedures, cartilage reconstruction, bone graft substitutes; hemostasis; wound treatment and management; reinforcement and support of tissues; incontinence; etc.

In one aspect, the present invention provides a biomaterial comprising collagen, wherein the biomaterial has a surface area greater than about 2.3 m²/g collagen. In other aspects, the biomaterial has a surface area selected from the group consisting of a surface area of or greater than about 2.5, 2.75, 3.0, 3.25, 3.5, 3.75, 3.8, 4.0, and 4.4 m²/g collagen. In a particular aspect, the invention encompasses a biomaterial comprising collagen, wherein the biomaterial has a surface area of or greater than about 4.0 m²/g collagen. In further aspects, the collagen is human collagen, recombinant collagen, recombinant human collagen, and collagen type I, respectively. In a preferred aspect, the collagen is recombinant human type I collagen. In some embodiments, the collagen is selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII.

A biomaterial comprising collagen, wherein the biomaterial has a surface area of or greater than about 4.0 m²/g collagen, is provided. In certain embodiments, the collagen is recombinant collagen, human collagen, recombinant human collagen, and type I collagen, respectively. In a particular embodiment, the collagen is recombinant human collagen type I.

A biomaterial comprising collagen, wherein the biomaterial has a surface area of or greater than about 3.8 m²/g collagen, is also provided. In certain embodiments, the collagen is recombinant collagen, human collagen, recombinant human collagen, and type II collagen, respectively. In a particular embodiment, the collagen is recombinant human collagen type II.

A biomaterial comprising collagen, wherein the biomaterial has a surface area of or greater than about 4.4 m²/g collagen, is also provided. In certain embodiments, the collagen is recombinant collagen, human collagen, recombinant human collagen, and type III collagen, respectively. In a particular embodiment, the collagen is recombinant human collagen type III.

In one aspect, the present invention provides a biomaterial comprising human collagen, wherein the biomaterial has an average pore size of less than about 40 μm. In a certain aspect, the human collagen is recombinant human collagen. In some embodiments, the collagen is selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII.

A biomaterial comprising human collagen and having an average pore size of about 35 μm is specifically provided. In one aspect, the collagen is recombinant human collagen. In a further aspect, the human collagen is type I collagen. In a preferred aspect, the human collagen is recombinant human collagen type I.

A biomaterial comprising human collagen and having an average pore size of about 32 μm is also provided. In one aspect, the collagen is recombinant human collagen. In a further aspect, the human collagen is type II collagen. In a preferred aspect, the human collagen is recombinant human collagen type II.

A biomaterial comprising human collagen and having an average pore size of about 28 μm is additionally provided. In one aspect, the collagen is recombinant human collagen. In a further aspect, the human collagen is type III collagen. In a preferred aspect, the human collagen is recombinant human collagen type III.

In various embodiments, the invention provides biomaterials comprising human collagen and having a pore size range of from about 10 to 55 μm. In certain embodiments, the human collagen is recombinant collagen, and, in further embodiments, the human collagen is recombinant human collagen type II and recombinant human collagen type III. A biomaterial comprising human collagen and having a pore size range of from about 15 to 60 μm is also contemplated. In certain embodiments, the human collagen is recombinant collagen, and, in further embodiments, the human collagen is recombinant human collagen type I.

In one aspect, the invention encompasses a biomaterial comprising recombinant collagen, wherein the biomaterial has a tensile strength of greater than about 1.5 N. In some embodiments, the collagen is selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII. In another aspect, the biomaterial is a membrane or sheet. The invention further provides biomaterials comprising collagen and having tensile strengths of or greater than about 2.0, 2.5, 3.0, 3.5, 4.0, 4.5, 5.0, and 5.5 N.

In a particular embodiment, the invention encompasses a biomaterial comprising collagen, wherein the biomaterial has a tensile strength of about 4.0 N. In further embodiments, the collagen is human collagen, recombinant collagen, recombinant human collagen, or collagen type III, respectively. In a certain embodiment, the collagen is recombinant human collagen type III.

In one aspect, the invention provides a biomaterial comprising collagen, wherein the biomaterial has a tensile strength of about 0.1333 N/mm³. In another aspect, the invention provides a biomaterial comprising collagen and having a tensile strength greater than about 0.0088 N/mm³. In a further aspect, the invention provides a biomaterial comprising collagen, wherein the biomaterial has a tensile strength of or greater than about 0.01, 0.02, 0.03, 0.04, 0.05, 0.06, 0.07, 0.08, 0.09, 0.10, 0.11, 0.12, and 0.13 N. In various aspects, the collagen is human collagen, recombinant collagen, recombinant human collagen, or collagen type III, respectively. In a particular aspect, the collagen is recombinant human collagen type III.

Biomaterials comprising collagen, wherein the biomaterial has a tensile strength of or greater than about 5.8 N, are provided. In various aspects, the collagen is human collagen, recombinant collagen, recombinant human collagen, or collagen type I, respectively. In a particular aspect, the collagen is recombinant human collagen type I.

Biomaterials comprising collagen, wherein the biomaterial has a tensile strength of or greater than about 1.2 N are also provided. In various aspects, the collagen is human collagen, recombinant collagen, recombinant human collagen, or collagen type I, respectively. In a particular aspect, the collagen is recombinant human collagen type I.

In one aspect, the invention provides a biomaterial comprising recombinant human collagen, wherein the biomaterial has a denaturation temperature of greater than about 36.9° C. The invention further encompasses a biomaterial comprising recombinant human collagen, wherein the biomaterial has a denaturation temperature greater than about 37° C., 37.3° C., 40° C., 42° C., 50° C., and 55° C. In various aspects, the collagen is human collagen, recombinant collagen, recombinant human collagen, etc. In a particular aspect, the collagen is recombinant human collagen type I.

The invention further provides a biomaterial comprising collagen, wherein the biomaterial is collagenase-resistant. For purposes of the present invention, a biomaterial that is “collagenase-resistant” is a biomaterial in which greater than about 10% of the collagen in that biomaterial remains, e.g., is undigested or not degraded, after exposure to collagenases for a specific period of time. Therefore, in one aspect, the present invention provides a biomaterial comprising collagen, wherein the biomaterial is collagenase-resistant. In preferred aspects, the biomaterial is a membrane or a sheet.

In various embodiments, the biomaterial is human collagen, recombinant collagen, or recombinant human collagen, respectively. In some embodiments, the collagen is selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII. The collagen can be collagen of one type free of any other type, or can be a mixture of collagen types.

In certain embodiments, the biomaterial has a degree of collagenase resistance selected from the group consisting of greater than about 10%, greater than about 20%, greater than about 30%, greater than about 40%, greater than about 50%, greater than about 60%, or greater than about 70% collagenase resistance. In other embodiments, the biomaterial has a degree of collagenase resistance selected from the group consisting of greater than about 80%, greater than about 85%, greater than about 90%, greater than about 95%, greater than about 96%, greater than about 97%, greater than about 98%, greater than about 99%, or about 100% collagenase resistance. In preferred embodiments, the biomaterial is a membrane or a sheet. In various embodiments, the collagen is human collagen, recombinant collagen, or recombinant human collagen.

A method for preparing a biomaterial comprising recombinant human collagen is provided, the method comprising: (a) providing recombinant human collagen monomers; (b) forming recombinant human collagen fibrils comprising the recombinant human collagen monomers; (c) crosslinking the recombinant human collagen fibrils to form recombinant human collagen oligomers; (d) crosslinking the recombinant human collagen oligomers in a mold; and (e) lyophilizing the material to form a biomaterial comprising recombinant human collagen. The invention further provides a biomaterial prepared according to the above-described method.

### BRIEF DESCRIPTION OF THE DRAWINGS

FIGS. 1A and 1B show scanning electron micrograph analysis of collagen biomaterials.

FIGS. 2A and 2B show microscopic analysis of collagen membrane biomaterials.

FIGS. 3A and 3B show resistance of collagen membrane biomaterials to bacterial collagenase digestion.

FIG. 4 shows resistance of collagen membrane biomaterials to mammalian collagenase digestion.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to a “compound” is a reference to one or more compounds and to equivalents thereof as described herein and as known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention,

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Phanrmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R, and Graham, A., eds. (1997) PCR (Introduction to Biotechniques Series), 2nd ed., Springer Verlag.

Invention

The present invention provides compositions containing and methods for formulating biomaterials comprising collagen, such biomaterials being appropriate for use in various medical applications and devices. These biomaterials include, e.g., sponges, matrices, membranes, sheets, hemostats, dressings, antimicrobial dressings, scaffolds, barriers, stents, tissue grafts, tissue and vascular sealants, corneal shields, artificial skin, implants, coatings, adhesives, sutures, etc., containing collagens. In particular, the present invention relates to biomaterials possessing unique microstructures and specific architectural characteristics. These physical parameters result in materials and devices possessing properties distinct from those attainable by currently available devices, including, e.g., enhanced surface area, greater tensile strength, higher denaturation temperature, dramatically increases resistance to degradation by collagenases, etc.

In one embodiment, the biomaterials of the present invention have a high surface area. These biomaterials provide, for example, enhanced loading capacity for drugs and biologics. Biomaterials of the present invention, and devices containing them, thus provide for better control and improved release kinetics of drug and biologics. Drugs or biologics that adhere to the surface of the delivery vehicle can be presented to cells binding to the surface. For example, DNA gene constructs deposited on biomaterials of the present invention are taken up by cells that have subsequently bound to the biomaterial. With a larger surface area, increased amounts of drug and biologics can be added to the biomaterial, as more cells can bind, interact with, and uptake a greater amount of drug or biologic contained within the biomaterial. A high surface area is also beneficial in allowing for enhanced control over the rate of diffusion of drugs and biologics contained within the biomaterial into the extracellular fluid and presenting more contact surface with the surrounding environment. Thus, the ability to produce materials specifically designed to enhance surface area allows for the development of biomaterials engineered for optimal therapeutic effect.

In another embodiment, the enhanced surface area of the biomaterials of the present invention provides for enhanced cell-matrix (e.g., collagen) interactions, resulting in increased cell proliferation, migration, differentiation, and survival. Interaction of cells with various collagens is mediated by two classes of receptors on cell surfaces: integrins (Heino (2000) Matrix Biol 19:319-323) and discoidin domain receptors (Vogel (2001) Eur J Derm 11:506-514). Higher surface area, as provided by the biomaterials of the present invention, enables the presentation of collagen molecules accessible to these receptors, permitting increased interaction with cells, and leading to corresponding effects on cell physiology and function, including, for example, enhanced cell attachment, proliferation, differentiation, and survival. Therefore, in certain aspects, the present devices provide enhanced performance in, for example, wound healing and tissue engineering applications.

In one aspect, the present invention provides biomaterials having homogeneous microstructure containing thin collagen matrix sheets and interconnected pores. This structure provides increased permeability into the biomaterial, thus facilitating diffusion of nutrients to and waste from cells within or associated with the biomaterial. In further aspects, biomaterials of the present invention have high tensile strength and improved structural and mechanical integrity.

Biomaterials of the present invention are collagenase resistant, e.g., resistant to digestion by bacterial or mammalian collagenases. Collagenase resistant biomaterials provide more effective and long-lasting barriers for various medical applications, such as, for example, enhanced or guided tissue regeneration.

“Collagenase resistance” refers to the ability of a biomaterial to resist degradation by collagenases. The degree of collagenase resistance can be expressed as the amount of collagen remaining after exposure to various collagenases, or as the amount of collagen degraded during that exposure. For purposes of this application, collagenase resistance was measured by exposure of test materials to specific collagenases for predetermined time periods. The percentage collagen remaining and degraded were measured.

The biomaterials of the present invention displayed a significantly high level of collagenase resistance in each test applied, including exposure to both bacterial and mammalian collagenases. Greater than 80%, and, in some cases, greater than 90% of the collagen contained in the biomaterials of the present invention remained after exposure to the collagenases t In contrast the commercially available bovine collagen membrane tested displayed a low level of collagenase resistance, as less than about 10% of the collagen contained in that material remained at the end of the exposure period.

For purposes of the present invention, a biomaterial that is “collagenase-resistant” is a biomaterial in which greater than about 10% of the collagen in that biomaterial remains, e.g., is undigested or not degraded, after exposure to collagenases for a specific period of time. Therefore, in one aspect, the present invention provides a biomaterial comprising collagen, wherein the biomaterial is collagenase-resistant. In preferred aspects, the biomaterial is a membrane or a sheet

In various embodiments, the biomaterial is human collagen, recombinant collagen, or recombinant human collagen, respectively. In some embodiments, the collagen is selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII. The collagen can be collagen of one type free of any other type, or can be a mixture of collagen types.

Preferably, the invention provides biomaterials having certain degrees of collagenase-resistance, e.g., wherein greater than 10%, greater than 20%, greater than 30%, greater than 40%, greater than 50%, greater than 60%, or greater than 70% of the collagen in these biomaterials remains after exposure of these biomaterials to collagenases for a specific period of time. In a preferred embodiment, the present invention provides biomaterials having a high degree of collagenase-resistance, e.g., biomaterials containing collagen wherein greater than 80%, greater than 85%, greater than 90%, greater than 95%, greater than 96%, greater than 97%, greater than 98%, greater than 99%, or 100% of the collagen contained in the biomaterial remains after exposure of the biomaterial to collagenases for a specific period of time.

The degree to which a biomaterial is collagenase-resistant can be expressed as the percentage collagen undigested or undegraded, e.g., remaining, after exposure to collagenases for a predetermined period of time. Thus, for example, a biomaterial of the present invention comprising collagen, wherein greater than 80% of the collagen contained in the biomaterial is remains after exposure to collagenases, is a biomaterial that is 80% collagenase-resistant; a biomaterial of the present invention comprising collagen, wherein greater than 90% of the collagen contained in the biomaterial is remains after exposure to collagenases, is a biomaterial that is 90% collagenase-resistant, and so on.

Collagenase resistance, e.g., resistance to digestion by bacterial or mammalian collagenase, can be readily determined by various methods well known to those of skill in the art. For example, in one method, resistance of a collagen composition or biomaterial to bacterial collagenase is determined as follows. The collagen composition or biomaterial is mixed in digestion buffer (110 mM NaCl, 5.4 mM KCl, 1.3 mM MgCl₂, and 0.5 mM ZnCl₂ in 21 mM TRIS, pH 7.45) at a ratio of 0.2 mls digestion buffer per 1 mg dry weight collagen. Bacterial collagenase (form III from Clostridium histolyticum) is added to the solution of collagen composition or biomaterial to a final concentration of 50 unites collagenase per 1 mg dry weight collagen. The mixture is incubated for 6 hours at 37° C. Following collagenase digestion, any remaining collagen composition or biomaterial is pelleted by centrifugation and both pellet and supernatant are retained. The collagen pellet is dissolved in 0.5 ml NaOH by heating at 70° C. for 30 minutes, followed by neutralization by addition of an equivalent amount of 0.5 M HCl. Protein concentrations of both the supernatant and solubilized pellet are determined using any standard protein determination assay, such as BCA assay. Total protein content of the supernatant (indicating digested material) and solubilized pellet (indicating collagenase resistant material) are determined and percent collagen undigested or remaining and or percent collagen digested or degraded is determined.

In another method, resistance of a collagen composition or biomaterial to a mammalian collagenase is determined as follows. The collagen composition or biomaterial is mixed in buffered solution (pH 7.0) with mammalian collagenase (e.g., matrix metalloprotease-1 (MMP-1) or matrix metalloprotease-8 (MMP-8)) at a ratio of about 0.5 μg mammalian collagenase to 2.0 to 2.5 mg dry collagen. The mixture is incubated at 37° C. for 1, 3, and 6 days. At each time point, a measured aliquot of the digestion mixture is centrifuged, and the protein concentration of the resulting supernatant is measured using any standard protein determination assay, such as BCA assay. Protein content of the supernatant indicates digested material percent collagen undigested or remaining and or percent collagen digested or degraded is determined.

In other aspects, the biomaterials of the present invention provide pure, homogeneous, and consistent material for various biomedical applications. The material can comprise one specified collagen type, or can comprise a specified mixture of collagen types. The recombinant human collagen biomaterials, e.g., membranes, matrices, etc., have unique and defined compositions and architectural structure. In one aspect, the present devices can be used, e.g., in applications involving three-dimensional printing and micro- and nano-patterning.

It is contemplated that the biomaterials of the present invention can include biomaterials in any one of the forms standard and widely used in the field, including, for example, sponges; matrices; membranes; sheets; implants; scaffolds; barriers; stents; grafts, e.g., a tissue graft; sealants, e.g., vascular sealants, tissue sealants, etc.; corneal shields; artificial tissues, e.g., artificial skin; hemostats; bandages; dressings, e.g., wound dressings coatings, e.g., stent coatings, graft coatings, etc.; adhesives; sutures; and drug delivery devices. It is further contemplated that the present biomaterials can be used in any of various applications and procedures, including, but not limited to, the following: tissue engineering, tissue augmentation, guided tissue regeneration; drug delivery; various surgical procedures including restorative, regenerative, and cosmetic procedures; vascular; osteogenic and chondrogenic procedures, cartilage reconstruction, bone graft substitutes; hemostasis; wound treatment and management; reinforcement and support of tissues; incontinence

Guided tissue regeneration, a surgical procedure for advanced cases of periodontal disease, treats defects located below the gumline. During GTR procedure, plaque is removed from the root of the tooth, and a barrier membrane is placed over the defect, guarding the cavity against tissue invasion, giving bone and ligaments sufficient time to regenerate. The performance of collagen membranes for GTR in dentistry depends on the membranes's ability to prevent epithelial cell growth and the membrane's resistance to bacterial collagenase digestion. The membrane biomaterial of the present invention is less porous than the commercial animal collagen BIOMEND absorbable collagen membrane currently used for GTR in dentistry, which may be more effective to prevent cell in-growth. The membrane biomaterials of the present invention are resistant to bacterial collagenase digestion. In the oral environment, bacterial collagenase may be involved in the degradation of collagen implants. The resistance to bacterial collagenase is an important performance parameter for the utility of a membrane biomaterial. A collagenase-resistant membrane biomaterial provides an effective barrier longer for greater regenerative results. The membrane biomaterials of the present invention are useful in various other medical applications, such as, for example, dural closures, wound dressings, reinforcement and support of weak tissues, etc.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### Preparation of Recombinant Human Collagen Fibrils

Recombinant human collagen is obtained, for example, as described in U.S. Pat. No. 5,593,859, incorporated by reference herein in its entirety. Recombinant human collagen type I, type II, and type III are listed herein by way of example, and the use of collagen of any type is clearly contemplated herein. Generally, the recombinant human collagen compositions and biomaterials described relate to production of oligomers from recombinant human collagen monomers and in-mold fibrillogenesis/cross-linking methods followed by lyophilization.

Recombinant human collagen type I, type II, or type III fibrils were prepared as follows. Fibrllogenesis buffer (0.2 M Na₂HPO₄, pH 11.2) was added to a 0.3% (3 mg/ml in 10 mM HCl) solution of recombinant human collagen type I, type II, or type III at a 1:10 (v/v) ratio. The solution was incubated at room temperature from 4 hours to overnight. Following fibrillogenesis, recombinant human collagen fibrils were then collected by centrifugation at 15,000×g for 30 minutes at 10° C.

### Example 2

### Preparation of Recombinant Human Collagen Oligomers

Recombinant human collagen oligomers were prepared from recombinant human collagen fibrils. The preparation of recombinant human collagen oligomers from recombinant human collagen monomers is also contemplated herein. Recombinant human collagen fibrils were prepared as described in Example 1 above. A 20% solution (w/v) of EDC (1-ethyl-3-(3-dimethylamino propyl)carbodiimide), prepared in water immediately before use, was added to a solution of recombinant human collagen fibrils to a final concentration of 0.15% EDC (for recombinant human collagen type I and type III fibrils) or 0.075% EDC (for recombinant human collagen type II fibrils). The solutions were mixed thoroughly and incubated at room temperature for 16 hours.

The resulting cross-linked recombinant human collagen fibrils (i.e., recombinant human collagen oligomers) were then centrifuged in a Beckman JA-14 rotor at 10,000 rpm (approximately 9,000×g) for 30 minutes at 20° C. in a Beckman J2-21m centrifuge. The supernatant was carefully removed by decanting into an Erlenmeyer flask The pellets were washed by resuspending them in water to their original volumes followed by vigorous agitation. The solution was centrifuged and the resulting supernatant removed as described above. The pellets were resuspended in water or 10 mM HCl to a final recombinant human collagen concentration of 30 mg/ml. The recombinant human collagen oligomer suspension in water was redissolved by adding 1/10 volume of 100 mM HCl to the collagen/water resuspension. The resulting recombinant human collagen oligomers were evaluated by SDS-PAGE on 4-20% polyacrylamide gradient gels, which showed that higher molecular weight oligomers of recombinant human collagen were produced. Recombinant human collagen type I, type I, and type III oligomers were successfully formed from recombinant human collagen type I, type II, and type monomers, respectively (data not shown).

### Example 3

### Rotary Shadowing Electron Microscopy of Recombinant Human Collagen Type I Monomers and Oligomers

Formation of recombinant human collagen oligomers was further confirmed by rotary shadowing electron microscopy. Recombinant human collagen type I oligomers, prepared as described in Example 2 above, were dialyzed against a solution of 50% glycerol in 0.05% acetic acid for 16 hours at 4° C. Following dialysis, samples were sprayed onto a freshly-cleaved mica substrate using an airbrush. The droplets on the mica were dried at room temperature at 106 mm Hg for 12 hours in a vacuum coater (Edwards 306). The dried samples were rotary shadowed with platinum using an electron gun positioned at 6° to the mica surface, and then coated with a film of carbon generated by an electron gun positioned at 90° to the mica surface. The replicas were floated on distilled water and collected on formvar-coated grids. The replicas were then examined on a Zeiss 109 transmission electron microscope.

Rotary shadowing electron microscopic analysis showed that the recombinant human collagen type I monomers displayed a fibrillar collagen morphology of rod shaped structures. Association of recombinant human collagen type I molecules into higher order oligomers was clearly visible. Similar results were observed with oligomers prepared from recombinant human collagen type II and type III. (Data not shown.)

### Example 4

### Preparation of Recombinant Human Collagen Type I, Type II, and Type III Matrices

A recombinant human collagen type III matrix was prepared as follows. Recombinant human collagen type III oligomers, prepared as described in Example 2 above, were resolubilized by addition of HCl to a final concentration of 10 mM HCl. Recombinant human collagen type III fibrils were reconstituted by addition of fibrillogenesis buffer at a 1:10 ratio (v/v), followed by cross-linking with EDC to a final concentration of 0.25% EDC. The solutions were incubated in stainless steel molds for 6 hours and then lyophilized using a Virtis Genesis 25EL lyophilizer.

A recombinant human collagen type I matrix was prepared as follows. Recombinant human collagen type I oligomers, prepared as described in Example 2 above, were mixed with 1/10 volume of 0.2 M NaH₂PO₄, pH 7.3, and 1/10 volume water. To this solution was added a freshly-prepared solution of 10% EDC in water, resulting in a final 20 mg/ml collagen concentration and 0.25% EDC. This solution was mixed well, transferred to stainless steel molds (3 mm in depth), and incubated at room temperature for 6 hours. The in-mold recombinant collagen type I matrix was then lyophilized at −30° C.

A recombinant human collagen type II matrix was prepared according to the protocol described above for recombinant human collagen type I and recombinant human collagen type III matrices with various modifications as follows. Briefly, a solution of recombinant human collagen type II was filtered using a 0.22 mm PES vacuum filter. The volume was calculated from the weight, and the collagen concentration adjusted to 3.0 mg/ml using 10 mM HCl. Fibrillogenesis buffer was added at a 1:10 (v/v) ratio. The pH of the collagen solution was determined and adjusted to pH 7.2 with 0.5 N NaOH, as necessary. Fibril formation was allowed to proceed for 6 hours at room temperature before cross-linker, 1-ethyl-3-(3-dimehtylaminopropyl) carbodiimide (EDC), was added to a final concentration of 0.075% (note that 0.15% EDC was used for preparation of recombinant human collagen type I and recombinant human collagen type III matrix). The collagen/cross-linker mixture was incubated overnight at room temperature.

Recombinant human collagen type II fibrils were pelleted by centrifugation using a Beckman JLA-16 rotor, 15,000 rpm for 60 minutes at 4° C. The supernatant was removed, and the pellet was washed with water using a volume equal to the original reaction volume. The recombinant human collagen type II fibrils were pelleted again by centrifugation. The supernatant was removed and saved for protein concentration determination by BCA. The pellet concentration was about 30 mg/ml. The cross-linked recombinant human collagen type II fibrils were dissolved in 10 mM HCl and formulated into a matrix following an in-mold fibrillogenesis/cross-linking method, as described above.

### Example 5

### Microstructure of Recombinant Human Collagen Type I Matrix by Histological Staining

The microstructure of a recombinant human collagen type I matrix was compared to that of INSTAT collagen absorbable hemostat (Ethicon, Inc., Somerville, N.J.), a commercially-available bovine collagen sponge, by microscopic examination of histologic sections stained with Congo Red under polarized light. (See, e.g., Sweat et al. (1964) Arch Pathol 78:69-72.) A 3 mm×3 mm recombinant human collagen type I matrix, prepared and lyophilized as described in Example 4 above, was rehydrated in distilled water for 15 to 30 minutes. The recombinant human collagen type I matrix was then dehydrated by sequentially incubating the matrix in a series of increasing alcohol concentrations (70%, 80%, 95%, 100%) for 15 minutes each with slow agitation. The recombinant human collagen type I matrix was then cleared in xylene for 15 minutes with slow agitation. The recombinant human collagen type I matrix was embedded in paraffin, cut at 5 μm thickness, stained with Congo Red, and observed under a microscope using polarized light.

The results showed a matrix formed from recombinant human collagen type I oligomers had a more intact and uniform porous network compared to INSTAT collagen absorbable hemostat. Similar microstructures were observed for recombinant human collagen type II and type III matrices. (Data not shown.)

### Example 6

### Scanning Electron Microscopic Analysis of Recombinant Human Collagen Type I Matrix

A recombinant human collagen type I matrix was examined using scanning electron microscopic (SEM) analysis. (See, e.g., Yang et al. (1994) Matrix Biology 14:643-651; Areida et al. (2001) J Biol Chem 276:1594-1601.) The recombinant human collagen type I matrix was frozen in liquid nitrogen for one minute and then cut with a cold razor blade. The resulting fractured recombinant human collagen type I matrix was mounted on standard SEM aluminum stubs (12 mm OD) with double-sided conductive tabs. The stubs with the samples were then sputter-coated with gold of 40 nm in thickness. (The coater, E5000M, S.E.M. made by Biorad Palaron Division, was used for these preparations.) The prepared stubs were characterized using the Personal SEM (ASPEX Instruments, Inc.) The structures of interest were photographed from 100× to 1000× magnification.

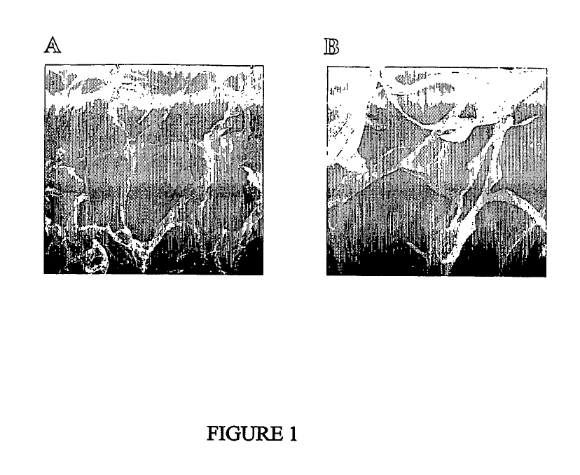

SEM analysis demonstrated distinct structural features of the recombinant human collagen type I matrix (FIG. 1A) compared to INSTAT collagen absorbable hemostat (FIG. 1B).

The recombinant human collagen type I matrix had a pore microstructure interconnected by thin sheets formed of recombinant human collagen filaments and fibrils. INSTAT collagen absorbable hemostat displayed much thicker and less uniform sheets and fibers. Recombinant human collagen type II and recombinant human collagen type III matrices showed morphology similar to that of the recombinant human collagen type I matrix (data not shown).

### Example 7

### Surface Area and Pore Size of Recombinant Human Collagen Type I, Type II, and Type III Matrices

Recombinant human collagen matrix preparations were characterized by determining total surface area and pore size using mercury porisometry. Mercury porisometry testing was performed by QuantaChrome Instuments (Boynton Beach, Fla.) using a PoreMaster33 mercury porisometer. Briefly, mercury intrusion was performed using a contact angle of 140° and an intrusion pressure range of 0.806 psi to 49.825 psi at 20° C. A low-pressure mercury intrusion method was performed on duplicate samples of each sponge. Mercury extrusion was performed over the range of 49.381 psi to 0.822 psi. Mercury intrusion and extrusion were monitored as a function of time, and the date was used to determine pore size using the Washburn equation. Sample weight was approximately 23 mg for each recombinant human collagen matrix, and approximately 15 mg for the INSTAT collagen absorbable hemostat.

Surface area was determined for matrices produced using recombinant human collagen type I, recombinant human collagen type II, recombinant human collagen type III, and INSTAT collagen absorbable hemostat, the results of which are shown below in Table 1.

TABLE 1

| | Surface Area | |
|---|---|---|
| Sample | (m²/g) | % increase over INSTAT |
| Recombinant human collagen | 4.0 | 74% |
| type I matrix | | |
| Recombinant human collagen | 3.8 | 65% |
| type II matrix | | |
| Recombinant human collagen | 4.4 | 91% |
| type III matrix | | |
| INSTAT | 2.3 | N/A |

As shown in Table 1 above, recombinant human collagen type I matrix, recombinant human collagen type II matrix, and recombinant human collagen type III matrix had higher surface area than that of INSTAT collagen absorbable hemostat. Total surface areas of matrices produced using recombinant human collagen were 3.8 m²/g or higher, whereas total surface area for INSTAT collagen absorbable hemostat was 2.3 m²/g.

Pore size was determined for matrices produced using recombinant human collagen type I, recombinant human collagen type II, recombinant human collagen type III, and INSTAT collagen absorbable hemostat, the results of which are shown below in Table 2.

TABLE 2

| | Pore Size |
|---|---|
| Sample | (μm) |
| Recombinant human collagen | 35 |
| type I matrix | |
| Recombinant human collagen | 32 |
| type II matrix | |
| Recombinant human collagen | 28 |
| type III matrix | |
| INSTAT | 40 |

As shown above in Table 2, recombinant human collagen type I matrix, recombinant human collagen type II matrix, and recombinant human collagen type III matrix had smaller pore size than that of INSTAT collagen absorbable hemostat. Pore sizes of matrices produced using recombinant human collagen were 35 μm or lower, whereas pore size for INSTAT collagen absorbable hemostat was 40 μm.

The range of pore sizes determined for recombinant human collagen type III matrix was approximately 10 to 55 μm, smaller than the range of pores sizes determined for INSTAT collagen absorbable hemostat, which was approximately 25 to 90 μm. The highest population of pore size for recombinant human collagen type III matrix was approximately 28 μm, while that for INSTAT collagen absorbable hemostat was approximately 40 μm. The range of pore sizes determined for recombinant human collagen type I matrix and recombinant human collagen type II matrix was 15 to 60 μm and 10 to 55 μm, respectively.

### Example 8

### Tensile Strength and Denaturation Temperature of Recombinant Human Collagen Matrices

Tensile strength and denaturation temperature of recombinant human collagen matrix preparations were determined. Tensile strength was determined indirectly using a Texture Analyzer. Tensile strength, in Newtons (N), of recombinant human collagen type III and INSTAT collagen absorbable hemostat are shown below in Table 3.

TABLE 3

| | Tensile Strength | Tensile Strength |
|---|---|---|
| Sample | (N) | (N/mm³) |
| Recombinant human collagen | 4.0 +/− 0.2 | 0.1333 |
| type III matrix | | |
| INSTAT | 1.5 +/− 0.5 | 0.0088 |

As shown above in Table 3, recombinant human collagen type III matrix had a higher tensile strength (4.0+/−0.2 N) than that of INSTAT collagen absorbable hemostat (1.5+/−0.5 N). Tensile strength for each matrix was normalized to area (mm³), the results of which are shown above in Table 3. The data showed that recombinant human collagen matrix had a tensile strength of 0.1333 N/mm³, whereas the tensile strength of INSTAT collagen absorbable hemostat was 0.0088 N/mm³.

Tensile strength of recombinant human collagen type I membranes (prepared as described in Example 10 below) was determined indirectly using a Texture Analyzer. The recombinant human collagen type I membranes were first cross-linked with formaldehyde vapor (37% formaldehyde solution under vacuum for 60 minutes, room temperature) before use. The recombinant human collagen type I membranes were cut into 5 mm×20 mm pieces, either dry or wetted with water for 30 minutes, a vicryl 6.0 suture was attached, and the tensile strength was tested. The results are shown below in Table 4.

TABLE 4

| | Tensile Strength |
|---|---|
| Sample | (N) |
| Recombinant human collagen | 5.8 +/− 0.4 |
| type I membrane (dry) | |
| Recombinant human collagen | 1.2 +/− 0.2 |
| type I membrane (wetted) | |

As shown in Table 4 above, dry recombinant human collagen type I membrane had a tensile strength of 5.8+/−0.4 N, while wetted recombinant human collagen type I membrane had a tensile strength of 1.2+/−0.2 N. The wetting expansion (thickness) of recombinant human collagen type I membranes was also determined. The results showed that the wetted membrane expanded 50-60% (data not shown).

Denaturation temperature (T_{d}) was determined for recombinant human collagen type I matrix and recombinant human collagen type II matrix. Denaturation temperature for recombinant human collagen type II matrix was tested either dry or in solution (30 μl of PBS). Denaturation temperature was determined by heating each sample from 25° C. to 90° C., using a 5° C. per minute heating rate in a dry nitrogen environment. The results of these experiments are shown below in Table 5.

TABLE 5

| | Denaturation |
|---|---|
| | Temperature (T_{d}) |
| Sample | (° C.) |
| Recombinant human collagen | 58.1 |
| type I matrix (dry) | |
| Recombinant human collagen | 56.6 |
| type II matrix (wetted) | |
| (sample 1) | |
| Recombinant human collagen | 58.9 |
| type II matrix (wetted) | |
| (sample 2) | |
| Recombinant human collagen | 56.9 +/− 0.1 |
| type III matrix | |
| INSTAT (dry) | 36.9 +/− 0.4 |
| INSTAT (wetted) | 40.2 |

These results showed that recombinant human collagen matrices and biomaterials of the present invention had higher denaturation temperatures than that of INSTAT collagen absorbable hemostat.

### Example 9

### Assay for Cell Attachment on Recombinant Human Collagen Type I Matrix

Experiments were performed for evaluating cell attachment on recombinant human collagen type I matrices as described below. A recombinant human collagen type I matrix of the present invention was cut in half. The recombinant human collagen type I matrix was soaked in serum free media (DMEM with 4.5 mg/ml glucose) and equilibrate in 37° C. for 1 hour, and the media was changed once. The recombinant human collagen type I matrix was cut using a 3 mm biopsy puncher and patted dry with a sterile filter. The recombinant human collagen type I matrix was not allowed to dry out during these procedures. Human foreskin fibroblast cells used for the assay were trypsinized, counted, washed two times in serum-free media, and diluted to a final concentration of either 5 million per ml or 2.5 million per ml. The semi-dry recombinant human collagen type I matrices were placed in a 3 mm sterile petri dish, and the cells were loaded from the same edge of the recombinant human collagen type I matrix. The cells were allowed to attach for 2 hours at 37° C.

Another tissue culture plate containing a known amount of cells in a serum-free media was prepared and incubated at 37° C. Four milliliters of serum-free media was added to the 3 mm plates and the plates incubated and mixed slowly in a circular motion, which rinsed off any unattached cells from the recombinant human collagen type I matrices. Unattached and dead cells were removed by aspirating the media. Each recombinant human collagen type I matrix was gently transferred to a fresh tissue culture plate (1 matrix per well) using a pipette tip. To each well was added 200 μl of serum-free media and 20 μl of WST. The plates were incubated for 30 minutes, 1 hour, and 2 hours.

The plates were gently and lightly tapped, and a 110 μl aliquot from each tissue culture well was added into a new tissue culture plate. Absorbances were read at 450 nm. After measuring the absorbances, the 110 μl aliquot was transferred back to the original plate and incubated further for other time-points.

A recombinant human collagen type I matrix was tested as three-dimensional scaffold to support human foreskin fibroblast adhesion. The recombinant human collagen matrix was seeded according to the following groups: low-density, containing 5×10⁴ cells per matrix; middle-density, containing 1×10⁵ cells per matrix; and high-density, containing 2×10 cells per matrix. The results indicated that the recombinant human collagen type I matrix was not saturated with cells, even at the highest cell density tested (2×10⁵ cells per matrix).

### Example 10

### Formulation of Recombinant Human Collagen Type I Membranes

Recombinant human collagen type I membranes were prepared as follows. In a JA14 centrifuge bottle, fibrillogenesis buffer (0.2M NaH₂PO₄, pH 11.2) and recombinant human collagen type I were mixed in 10 mM HCl at a 1:10 ratio using a serological pipet. This solution mixture was incubated at room temperature for 4 hours for fibril formation. A 20% EDC (1-ethyl-3-(3-dimethylamino propyl)carbodiimide) solution (in water) was prepared just prior to addition to the solution containing recombinant human collagen type I fibrils. The EDC solution was added to the recombinant human collagen type I fibril solution to a final EDC concentration of 0.15%, mixed thoroughly, and incubated at room temperature overnight.

The mixture was centrifuged at 10,000 rpm for 30 minutes at 20° C. in a Beckman J2-21M centrifuge. The supernatant was removed by carefully decanting it into an Erlenmeyer flask. The pellets were resuspended in water to their original volume and mixed by vigorous agitation. The solutions were centrifuged and resulting supernatants decanted as described above. The pellets were resuspended in water to a final recombinant collagen concentration of 30 mg/ml. To the pellet resuspension was added 1/10 volume of 100 mM HCl and 1/10 volume of water, and the solution mixed well. To the pellet solution was added 1/10 volume of 0.2M NaH₂PO₄, pH 7.3, and 1/10 volume of water, and the solution mixed well. Freshly prepared 10% EDC and water was added to the solution to adjust the recombinant collagen concentration to 20 mg/ml and the final EDC concentration to 0.25%. The recombinant collagen solution was then transferred to stainless steel molds (3 mm in depth), air dried at room temperature, and the EDC cross-linking by-product removed by washing with 70% ethanol.

The recombinant human collagen type I membrane obtained was approximately 100 μnm thick and contained about 6 mg/cm² of recombinant human collagen type I. The recombinant human collagen type I membrane maintained its physical integrity after incubation at 37° C. in PBS overnight

### Example 11

### Characterization of Recombinant Human Collagen Type I Membranes

The microstructure of a recombinant human collagen type I membrane and BIOMED absorbable collagen membrane (Sulzer Calcitek, Inc., Carlsbad, Calif.), a commercial bovine collagen membrane, was examined by histological analysis after processing using the following procedure. A 3 mm×3 mm recombinant human collagen type I membrane, prepared as described above in Example 10, was rehydrated in distilled water for 15 to 30 minutes and then dehydrated using a series of sequential incubations with 70%, 80%, 95%, and 100% ethanol for 15 minutes each with slow agitation, following by clearing in xylene for 15 minutes with slow agitation. The recombinant human collagen type I membrane was embedded in paraffin, cut to 5 μm thickness, stained with H&E, and observed under a microscope.

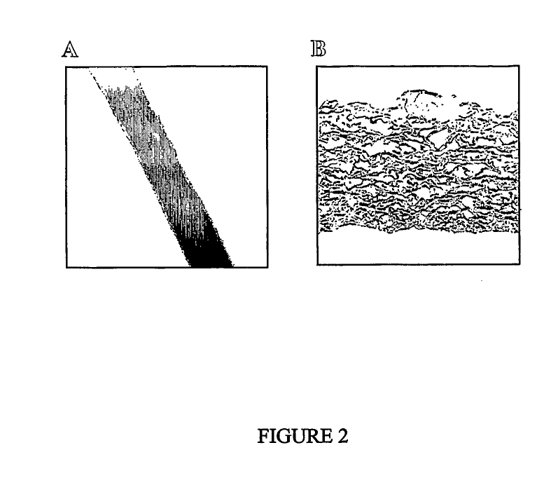

As shown in FIGS. 2A and 2B, recombinant human collagen type I membrane prepared using recombinant human collagen type I oligomers formed tightly packed filaments in an orientation parallel to the surface of the membrane (FIG. 2A), compared to BIOMEND absorbable collagen membrane (FIG. 2B).

### Example 12

### Resistance of Recombinant Human Collagen Type I Membranes to Bacterial Collagenase

The persistence of collagen-based biomaterials, such as matrices and membranes, can be correlated with their resistance to enzymatic digestions by proteinases, in particular, digestion by collagenase. The collagenase-resistance of recombinant human collagen type I membrane prepared by the processes described above was compared to that of BIOMEND absorbable collagen membrane.

A recombinant human collagen type I membrane of about 2.0 to 2.5 mg was added to a pre-weighed 0.5 ml microcentrifuge tube. A digestion buffer (110 mM NaCl, 5.4 mM KCl, 1.3 mM MgCl₂, and 0.5 mM ZnCl₂ in 21 mM Tris, pH 7.45) was added to the sample at a ratio of 0.2 ml per 1 mg dry recombinant human type I collagen. Bacterial collagenase (form m from Clostridiuin histolyticum) was added to the recombinant human collagen type I membrane to a final concentration of 50 units per mg dry collagen. Buffer only was added to the control samples. Samples were incubated for 6 hours at 37° C.

The remaining collagen was pelleted and the supernatant collected by centrifugation. The collagen pellet was dissolved in 0.5 mL of 0.5 M NaOH by heating at 70° C. for 30 minutes. The pellet was neutralized by adding an equal amount of 0.5 M HCl. Protein concentrations of both the supernatants and pellets were determined by BCA assay. Total protein content was calculated from these results, and the percent digestion was determined.

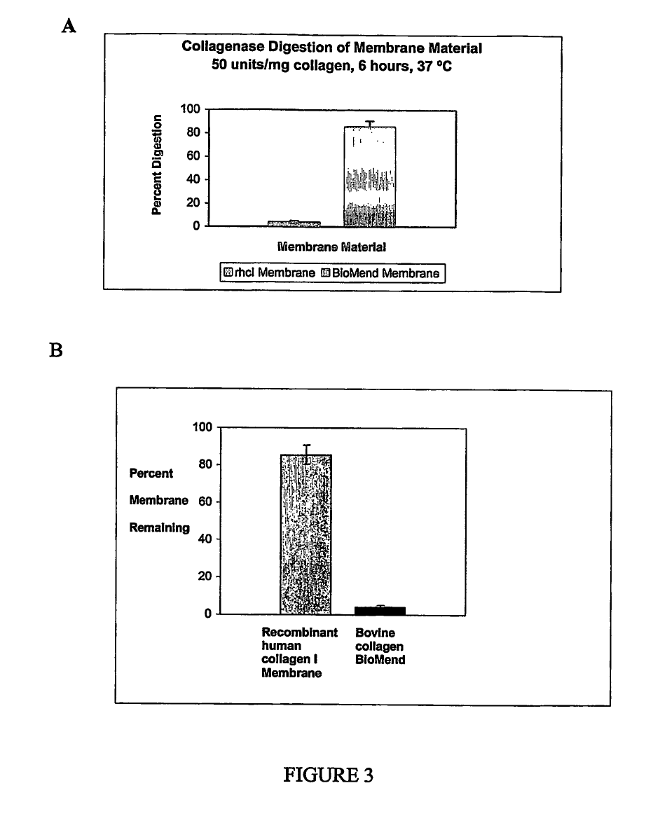

As shown in FIGS. 3A and 3B, recombinant human collagen type I membrane was more resistant to collagenase digestion than BIOMED absorbable collagen membrane. Less than 15% of the recombinant human collagen type I membrane was digested by bacterial collagenase in the assay used, compared to that of BIOMEND absorbable collagen membrane, of which greater than 80% of the membrane was digested by bacterial collagenase. Superior resistance to bacterial collagenase indicated that recombinant human collagen type I membranes provide an effective and longer-lasting membrane for various applications.

### Example 13

### Resistance of Recombinant Human Collagen Type I Membranes to Mammalian Collagenase

A comparison of mammalian collagenase resistance of a recombinant human collagen type I membrane to BIOMEND absorbable collagen membrane was performed. Briefly, 2.0 to 2.5 mg of dry collagen material was incubated with 0.5 μg mammalian collagenase (either MMP-1 or MMP-8) at 37° C. in buffer at pH 7.0. Aliquots were removed at days 1, 3, and 6. The collagen concentration of the supernatant was determined by BCA assay. Duplicate reactions were performed.

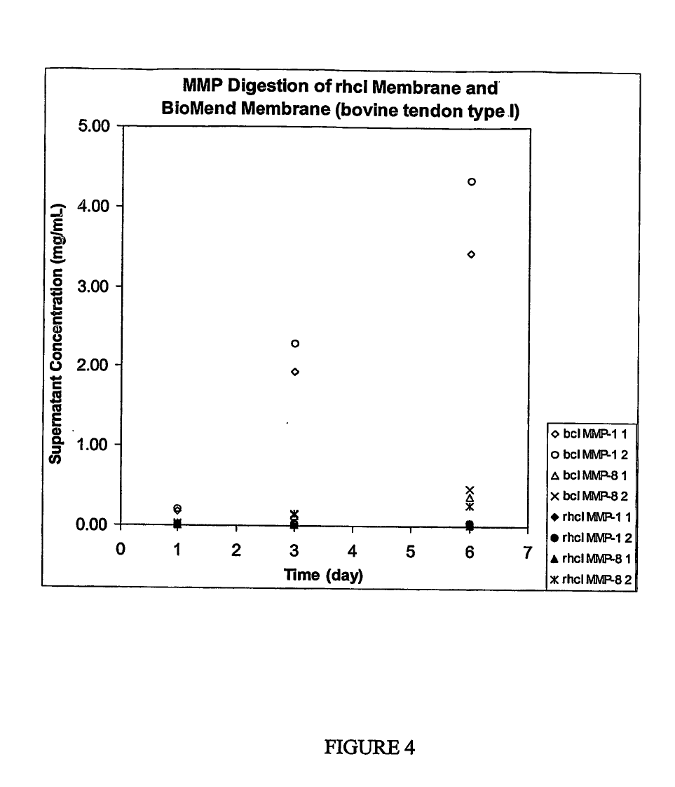

FIG. 4 shows the results of these experiments, plotted as protein concentration in supernatant as a function of time. As shown in FIG. 4, recombinant human collagen type I membrane (rhcI) exhibited higher resistance to digestion by both MMP-1 and MMP-8 compared to BIOMEND absorbable collagen membrane (bcI). By day 6, no BIOMED absorbable collagen membrane was visible in the MMP-1 digestion reaction solution. These results indicated that recombinant human collagen type I membrane had superior resistance to mammalian collagenase digestion. Therefore, membranes produced using recombinant human collagen are collagenase-resistant and long-lasting compositions, and provide more effective barriers useful in various applications, such as, for example, in guided tissue regeneration in dentistry.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

1. A biomaterial comprising a collagen of one collagen type free of any other collagen type, wherein the collagen type is selected from the group consisting of human collagen type I, human collagen type II, and human collagen type III, and further wherein the biomaterial has a surface area of greater than about 2.3 m²/g collagen.

2. The biomaterial of claim 1, wherein the biomaterial has a surface area of about 4.0 m²/g collagen.

3. The biomaterial of claim 2, wherein the collagen type consists of human type I collagen.

4. The biomaterial of claim 1, wherein the biomaterial has a surface area of about 3.8 m²/g collagen.

5. The biomaterial of claim 4, wherein the collagen type consists of human type II collagen.

6. The biomaterial of claim 1, wherein the biomaterial has a surface area of about 4.4 m²/g collagen.

7. The biomaterial of claim 6, wherein the collagen type consists of human type III collagen.

8. A biomaterial comprising a collagen of one collagen type free of any other collagen type, wherein the collagen type is selected from the group consisting of human collagen type I, human collagen type II, and human collagen type III, and further wherein the biomaterial has an average pore size of less than about 40 μm.

9. The biomaterial of claim 8, wherein the biomaterial has an average pore size of about 35 μm.

10. The biomaterial of claim 9, wherein the collagen type consists of human type I collagen.

11. The biomaterial of claim 8, wherein the biomaterial has an average pore size of about 32 μm.

12. The biomaterial of claim 11, wherein the collagen type consists of human type II collagen.

13. The biomaterial of claim 8, wherein the biomaterial has an average pore size of about 28 μm.

14. The biomaterial of claim 13, wherein the collagen type consists of human type III collagen.

15. A biomaterial comprising a collagen of one collagen type free of any other collagen type, wherein the collagen type is selected from the group consisting of human collagen type I, human collagen type II, and human collagen type III, and further wherein the biomaterial has a tensile strength of greater than about 1.5 N.

16. The biomaterial of claim 15, wherein the biomaterial is selected from the group consisting of a membrane and a sheet.

17. A biomaterial comprising a collagen of one collagen type free of any other collagen type, wherein the collagen type is selected from the group consisting of human collagen type I, human collagen type II, and human collagen type III, and further wherein the biomaterial has a tensile strength of greater than about 0.0088 N/mm³.

18. A biomaterial comprising a collagen of one collagen type free of any other collagen type, wherein the collagen type is selected from the group consisting of human collagen type I, human collagen type II, and human collagen type III, and further wherein the biomaterial has a degree of collagenase resistance of greater than about 10%.

19. The biomaterial of claim 18, wherein the biomaterial is selected from the group consisting of a membrane and a sheet.

20. A biomaterial comprising a collagen of one collagen type free of any other collagen type, wherein the collagen type is selected from the group consisting of human collagen type I, human collagen type II, and human collagen type III, and further wherein the biomaterial has a denaturation temperature of greater than about 36.9° C.
